(19) United States
(12) Patent Application Publication
(10) Pub. No.: US 2014/0190487 A1
(43) Pub. Date: Jul. 10, 2014

(54) TRACHEAL TUBE WITH PILOT VALVE BALLOON GUARD

(71) Applicant: COVIDIEN LP, Mansfield, MA (US)

(72) Inventors: Seamus Maguire, Athlone (IE); Kamlesh Sethiya, Athlone (IE); Carol Kiernan, Mullingar (IE); Emmet Bolger, Athlone (IE)

(73) Assignee: COVIDIEN LP, Mansfield, MA (US)

(21) Appl. No.: 13/737,782

(22) Filed: Jan. 9, 2013

(51) Int. Cl. A61M 16/04

(52) U.S. Cl. CPC A61M 16/0434
USPC 128/207.15

(57) ABSTRACT

A pilot balloon assembly for a tracheal tube includes a pilot balloon guard for a pilot balloon. The pilot balloon guard includes an enclosure having walls that define an interior volume that is greater than an interior volume of a pilot balloon. The pilot balloon is received by the enclosure and inflates within the enclosure to indicate inflation of a cuff of the tracheal tube. The pilot balloon guard enclosure absorbs externally applied forces to prevent transfer of the externally applied forces to the pilot balloon.

### BACKGROUND

The present disclosure relates generally to the field of tracheal tubes and, more particularly, to a tracheal tube having a pilot valve balloon guard.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of situations exist in which artificial ventilation of a patient may be desired. For short-term ventilation or during certain surgical procedures, endotracheal tubes may be inserted through the mouth to provide oxygen and other gasses to a patient. For other applications, particularly when longer-term intubation is anticipated, tracheostomy tubes may be preferred. Tracheostomy tubes are typically inserted through an incision made in the neck of the patient and through the trachea. A resulting stoma is formed between the tracheal rings below the vocal chords. The tracheostomy tube is then inserted through the opening. In general, two procedures are common for insertion of tracheostomy tubes, including a surgical procedure and a percutaneous technique.

Such tubes may include an inner cannula, such as a reusable inner cannula, or a disposable inner cannula. The inner cannula may be disposed inside the tracheostomy tube and used as a conduit for liquids or gas being exchanged with the patient's lungs. The inner cannula may be removed for cleaning and for disposal of secretions without disturbing the placement of the tracheostomy tube. A connector is typically provided at an upper or proximal end where the tube exits the patient's airway, suitable for coupling the ventilator with the inner cannula. In some products, the inner cannula may be removed, cleaned, and reused. In other products, the inner cannula may be disposable, and a new inner cannula may then be positioned inside of the tracheal tube. By enabling the cleaning and/or replacement of the inner cannula, a ventilation circuit may be kept clean and free of secretions.

Further, an inflatable cuff is often disposed about the outer cannula and is inflated to seal the cuff against a patient's airway to aid in maintaining the tube in the desired location. The air pressure of the inflated cuff is typically managed to maintain the pressure in a desired range such that there is enough air pressure to maintain the seal against the patient's airway without over pressurizing the cuff. For example, certain products concurrently inflate the cuff and a pilot balloon located external to the patient to provide an indication to the clinician of the state of inflation of the cuff. Unfortunately, in certain instances, the pilot balloon may undergo accidental compression, thereby resulting in transfer of air from the pilot balloon to the cuff and an increased pressure in the cuff. Accordingly, one difficulty that arises in the use of tracheal tubes is maintaining the pressure in the cuff within the desired range and avoiding overpressurization of the cuff.

### BRIEF DESCRIPTION OF THE DRAWINGS

Various aspects of the disclosed techniques may become apparent upon reading the following detailed description and upon reference to the drawings in which:

FIG. 1 is a perspective view of an embodiment of a tracheal tube having a pilot balloon guard disposed about the pilot balloon;

FIG. 2 is a perspective view of an embodiment of the pilot balloon guard of FIG. 1 having venting apertures;

FIG. 3 is a perspective view of an embodiment of the pilot balloon guard of FIG. 2 in an open position;

FIG. 4 is a perspective view of an embodiment of a pilot balloon guard having walls with reinforcing ribs;

FIG. 5 is a perspective view of an embodiment of the pilot balloon guard of FIG. 1 formed as a two piece assembly; and

FIG. 6 is a perspective view of an embodiment of one piece of the two piece assembly of FIG. 5.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described in detail below, provided herein are tracheal tube systems including pilot balloon assemblies having a pilot balloon guard. For example, in presently contemplated embodiments, a pilot balloon guard may enclose a pilot balloon and provide a barrier between the pilot balloon and externally applied forces. As such, the pilot balloon guard may reduce or prevent the transfer of compressive outside forces to the pilot balloon during operation. The foregoing feature may offer distinct advantages over traditional tracheal tube systems that do not include a pilot balloon guard because the possibility of the undesired transfer of air from the pilot balloon to the cuff may be reduced or eliminated with presently disclosed designs. Still further, in certain embodiments, the pilot balloon guard may include additional features, such as a transparent or translucent appearance, one or more support ribs, one or more venting apertures, and so forth that provide additional benefits. For example, by providing a transparent or translucent enclosure, the pilot balloon may be guarded from externally applied forces, but the clinician may still be able to view the state of inflation of the pilot balloon. These and other features of presently contemplated embodiments are described in detail below.

In certain embodiments, the provided tracheal tube and pilot balloon assemblies may be disposable rather than reusable and may be capable of conveying gas to and from the patient, such as during medical situations that necessitate prolonged ventilation. As such, the devices and techniques provided herein may enable maintaining a bidirectional gas flow between the patient and an external ventilation device. Accordingly, the tracheostomy tube assemblies provided herein may be adapted to be inserted into the trachea via a surgical incision in the neck such that after insertion of the tube into the trachea, a portion of the tube remains outside the patient. This portion extends outwards from the neck and may connect the tracheostomy tube to a ventilator or other medical device. That is, the provided tracheostomy tube assemblies may be used in conjunction with auxiliary devices, such as airway accessories, ventilators, humidifiers, and so forth, which may cooperate with the tube assemblies to maintain airflow to and from the lungs of the patient. For example, the tracheal tubes may be coupled to an adapter or connector that is configured to couple the tracheostomy tube assemblies described herein to the desired auxiliary device.

Furthermore, although the embodiments illustrated and described herein are discussed in the context of tracheostomy tubes, it should be noted that presently contemplated embodiments may include a pilot balloon guard coupled to or capable of being coupled to any of a variety of airway devices that may utilize a pilot balloon, or any other type of inflatable indicator. For example, the mechanically deployable sensor may be coupled to an endotracheal tube, a Broncho-Cath™ tube, a specialty tube, or any other airway device having an inflatable indicator. Furthermore, as used herein, the term “tracheal tube” may include an endotracheal tube, a tracheostomy tube, a Broncho-Cath™ tube, a specialty tube, or any other airway device.

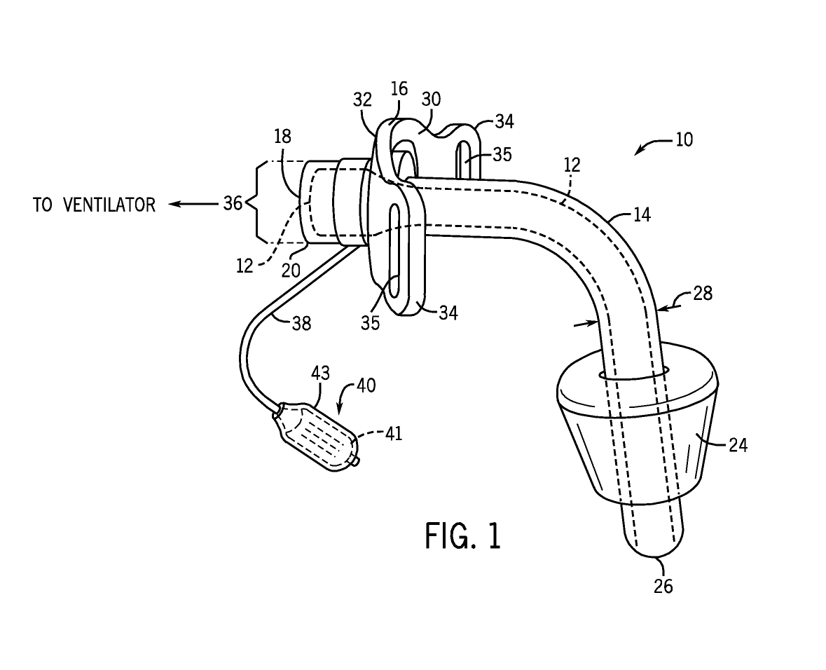

Turning now to the drawings, a tracheal tube consistent with one embodiment is illustrated in FIG. 1. As shown, a tracheal tube assembly 10 represented in the figures is a tracheostomy tube, although aspects of this disclosure could be applied to other airway devices or structures presenting the need for a balloon guard. In the illustrated embodiment, however, the tracheal tube assembly 10 includes a removable and/or disposable inner cannula 12 shown disposed inside of an outer cannula 14, which may be useful in maintaining a clean ventilation circuit. The outer cannula 14 is illustrated extending both distally as well as proximally from a flange member 16. The inner cannula 12 may be introduced through an opening 18 of an end connector 20 inside of the outer cannula 14.

During intubation, the tracheal tube assembly 10 is placed through an opening formed in the neck and trachea of a patient and extending into the patient's airway. The embodiment illustrated in the figures includes a sealing cuff 24, although in practice a wide range of tube designs may be used, including tubes having multiple cuffs around the outer cannula 14. The inner cannula 12 in the illustrated embodiment forms a conduit from which liquids or gases, including medications, may enter through the proximal opening 18 and exit through a distal opening 26. The cannula has an outer dimension 28 allowing it to fit easily through an incision made in the neck and trachea of the patient. In practice, a range of such tubes may be provided to accommodate the different contours and sizes of patients and airways. Such tube families may include tubes designed for neonatal and pediatric patients, as well as for adults. By way of example only, outer dimension 28 of the tube 20 may range from approximately 4 mm to approximately 16 mm in some embodiments.

In one embodiment, the outer cannula 14 enters the flange member 16 along a lower face 30 and protrudes through an upper face 32 of the flange member 16. When in use, the face 30 will generally be positioned against the neck of a patient, with the cannula extending through an opening formed in the neck and trachea. A pair of side wings or flanges 34 extend laterally and enable a strap or retaining member (not shown) to hold the tube assembly in place on the patient. In the illustrated embodiment, apertures 35 are formed in each side flange 34 to allow the passage of such a retaining device. In many applications, the flange member 16 may be taped or sutured in place as well.

In some embodiments, the outer cannula 14 may also include a suction lumen (not shown in FIG. 1) that extends from a location on the proximal end of the outer cannula 14 positioned outside the body when in use to a location around the cuff 24 inside the body. The suction lumen may terminate in a port through which secretions accumulated around the cuff may be aspirated. For example, a port may be located above the cuff 24 or one or more ports may be located anywhere along the length of the outer cannula 14 such that they aspirate secretions from the airway of the patient. Further, in some embodiments, an exterior suction tube may connect to the suction lumen for the removal of the suctioned fluids, for example, via a vacuum connected to the exterior suction tube.

The end connector 20 is formed in accordance with industry standards to permit and facilitate connection to a ventilation system. By way of example, standard outer dimensions may be provided as indicated at reference numeral 36 that enable a mating connector piece to be secured on the connector shown. By way of example, a presently contemplated standard dimension 36 accommodates a 15 mm connector, although other sizes and connector styles may be used. In use, then, air or other gas may be supplied through the connector and the inner cannula 12, and gases may be extracted from the patient.

For example, the tube assembly 10 may be inserted into the patient's airway, and the cuff 24 may then be inflated through an inflation lumen 38. In the illustrated embodiment, a pilot balloon assembly 40 includes a pilot balloon 41 that then indicates that air is in the cuff 24, thus sealing the patient's airway. The pilot balloon assembly 40 also includes a pilot balloon guard 43 that absorbs externally applied forces to protect the pilot balloon 41 from these forces. By reducing or eliminating the transfer of these forces to the pilot balloon 41, presently disclosed embodiments may reduce or eliminate the likelihood of air being transferred from the pilot balloon 41 to the cuff 24. The foregoing feature may, therefore, reduce or elimination the likelihood of the cuff 24 becoming overpressurized due to inadvertent or undesired compression of the pilot balloon 41. This may be particularly advantageous in embodiments in which the volume of air in the pilot balloon 41 approaches or equals the volume of air in the cuff 24, such as in pediatric or neonatal tube assemblies.

The pilot balloon guard 43 may be formed from a variety of types of materials and via a variety of types of processes. By way of example only, in some embodiments, the pilot balloon guard 43 may be formed via injection molding of a single-piece or multi-piece assembly. Further, the pilot balloon guard 43 may be made of any suitable material, such as polycarbonate, polyethylene, polypropylene, acrylonitrile butadiene styrene (ABS), rigid polyvinyl chloride (PVC), and so forth. Indeed, it should be noted that the manufacturing process and material utilized to make the pilot balloon guard 43 may be subject to a variety of implementation-specific considerations, such as but not limited to the size or type of tracheal tube with which the guard is designed to function, the reusability or disposability of the guard, and so forth. Still further, it should be noted that the pilot balloon guard 43 may be formed as a disposable device designed for a single use with a single tube, or the guard may be formed as a reusable device.

As mentioned above, the tracheal tube assembly 10 of FIG. 1 may, in some embodiments, be connected to a ventilator via a ventilation tube, thus enabling an assisted airway circuit through the patient's lungs. Additionally, it should be noted that the tracheal tube assembly 10 may be connected to other medical devices, such as a suction device, a T-junction, a medicine delivery system, and so forth, as desired according to implementation-specific considerations. Indeed, the end connector 20 may enable the attachment of one or more medical devices to the tracheal tube assembly 10.

Additionally, as appreciated by one skilled in the art, it should be noted that the components of the tracheal tube assembly 10 may be formed from various suitable materials via any appropriate manufacturing process. For example, the end connector 20 may be manufactured of materials such as a polyvinyl chloride (PVC), a PEBAX silicone, a polyurethane, thermoplastic elastomers, a polycarbonate plastic, a silicon, or ABS. For further example, the inner and/or outer cannulas 12 and 14 may be formed from a soft polyvinyl chloride (PVC) or another suitable plastic (e.g., polyurethane, thermoplastic elastomers, etc.) through an extrusion process. Still further, in certain embodiments, the end connector 20 and/or of the flange member 16 may be molded, overmolded, computer numerical control (CNC) machined, milled, or otherwise formed into the desired shape.

In certain embodiments, the illustrated components may be provided as a tube assembly kit and/or a pilot balloon assembly kit instead of in an assembled form. In embodiments in which a tube assembly kit is provided, the user or clinician may perform final assembly of the tracheal tube 10 by selecting a desired inner cannula 12 and then inserting the inner cannula 12 into the outer cannula 14 prior to intubation of the patient. More specifically, a distal end of the inner cannula 12 may be manually inserted inwardly into the outer cannula 14 through the opening 18.

Similarly, in some embodiments in which the pilot balloon assembly 40 is provided as a kit, the pilot balloon guard 43 may be selected from one of a variety of balloon guards based on the given application. In other embodiments, however, the pilot balloon guard 43 may be provided as a single component, for example, in a sterilized package. For further example, in certain embodiments, the pilot balloon guard 43 may be provided as a component in the tube assembly kit and, as such, may be preselected based on compatibility with the given tube. Indeed, the pilot balloon assembly 40 may include the pilot balloon guard 43, the pilot balloon 41, the inflation line 38, or any combination thereof, in addition to any other desired components, such as connectors or other coupling devices.

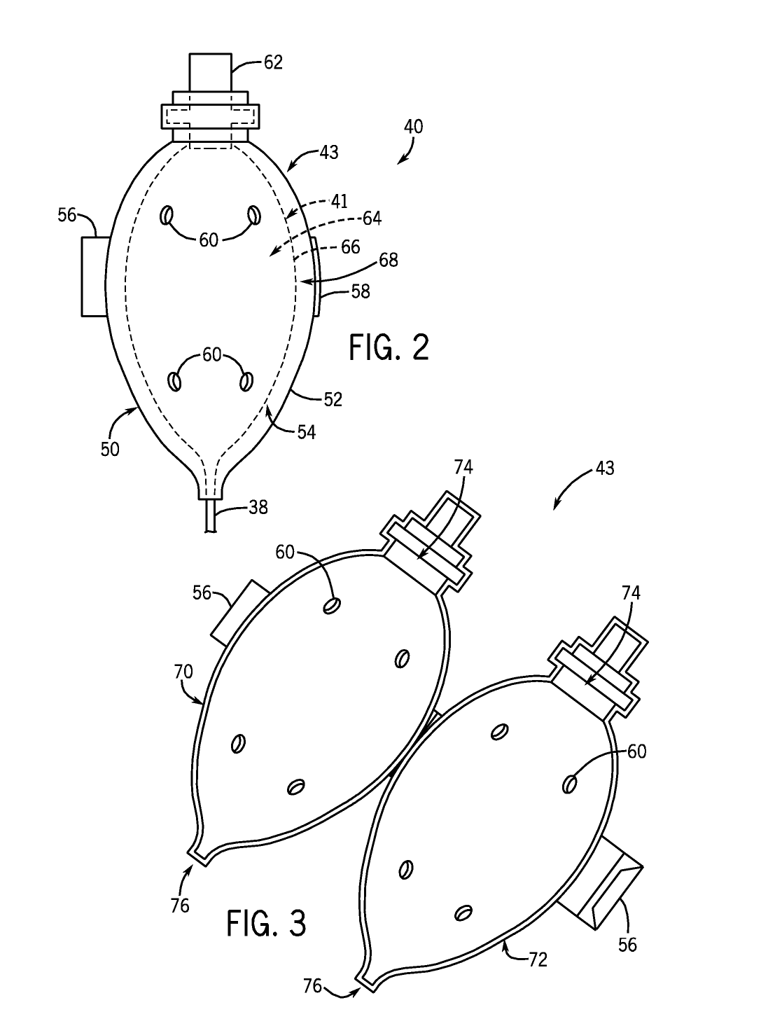

Turning now to FIG. 2, an embodiment of the pilot balloon assembly 40 is illustrated. In this embodiment, the pilot balloon guard 43 is formed as a clam shell type of enclosure 50 having walls 52 that define an interior volume 54 within the enclosure 50. The clam shell enclosure 50 also includes a clasp 56 and a hinge 58 to enable the pilot balloon guard 43 to be closed about the pilot balloon 41. Further, in the embodiment shown, the walls 52 of the enclosure 50 include apertures 60 that function as venting holes that enable transfer of air between the external environment and the interior volume 54, thus enabling equalization between the pressure within the volume 54 and atmospheric pressure.

Further, in this embodiment, the pilot valve balloon guard 43 is coupled to the pilot line 38 on one end and to a pilot line non-return valve 62 on the other end. In other embodiments, the valve 62 may be any standard valve coupled to the pilot line, such as a spring loaded non-return valve. Further, it should be noted that in other embodiments, the pilot balloon guard 43 may not be coupled to the pilot line 38 and the valve 62. Instead, the pilot valve balloon guard 43 may be secured about the pilot balloon 41 in any desired implementation-specific manner.

In the embodiment of FIG. 2, the pilot balloon 41 is positioned within the interior volume 54 of the enclosure 50. In some embodiments, the inflation capacity of a chamber 64 formed within walls 66 of the pilot balloon 41 may be less than the interior volume 54 of the pilot balloon guard enclosure 50. Accordingly, a chamber 68 may be formed between the walls 66 of the cuff 41 and the walls 52 of the enclosure 50. This chamber 68 may enable the pilot balloon 41 to inflate and deflate without limitations imposed by the enclosure 50, thus enabling the pilot balloon 41 to function as an indicator of the state of inflation of the cuff 24.

FIG. 3 illustrates the clam shell pilot balloon guard of FIG. 2 in an open position. Accordingly, as shown, the clasp 56 is unclasped, and a first clam shell half 70 and a second clam shell half 72 are coupled by the hinge 58. That is, in this embodiment, the pilot balloon guard 43 is made of two shells 70 and 72. Each shell 70 and 72 is hollow and concave to accommodate the pilot balloon 41 such that when the shells are positioned as in FIG. 2, the interior volume 54 is formed. Further, in the illustrated embodiment, a first end 74 of the assembly is sized and shaped to fit about a pilot valve, and a second end 76 of the assembly is sized and shaped to fit about a pilot line. However, in other embodiments, these features are subject to a variety of modifications based on the chosen way of attaching the guard to the tracheal tube assembly.

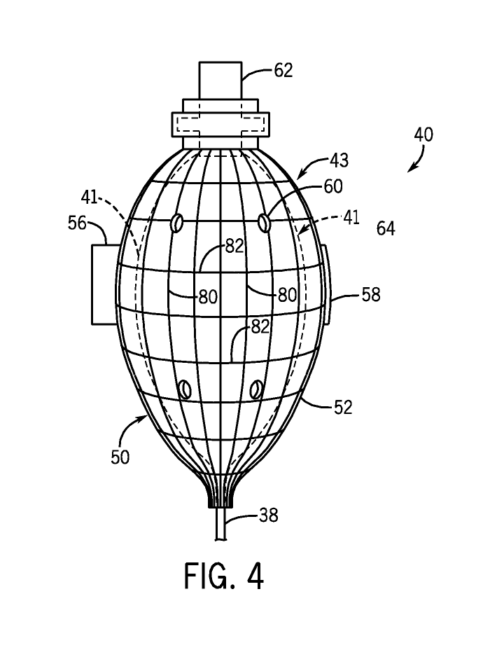

FIG. 4 illustrates a further embodiment of the pilot balloon guard 43 of FIG. 2. In this embodiment, the walls 52 of the enclosure 50 include a plurality of vertically disposed support ribs 80 and a plurality of horizontally disposed support ribs 82. However, it should be noted that in other embodiments, the quantity and type of support ribs may vary. In embodiments that include support ribs, the support ribs 80 and 82 may function to strengthen the walls 52 of the enclosure 50, thereby possibly enabling a thinner wall structure than non-reinforced walls in some embodiments. In certain embodiments, the wall structure 52 withstand forces up to between approximately 20-30 kg.

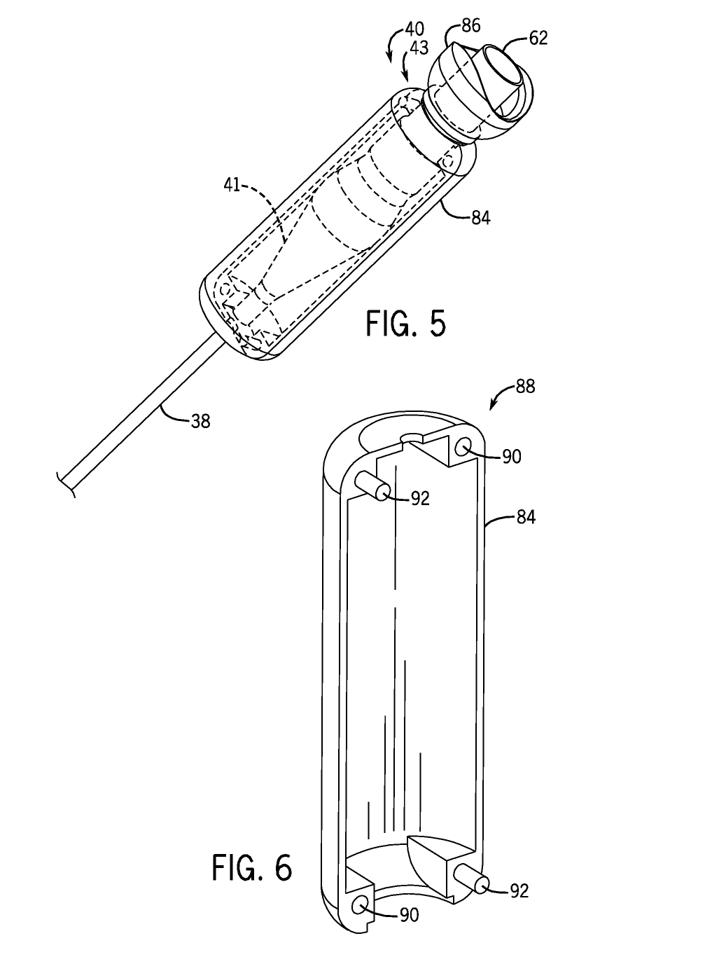

FIG. 5 illustrates another embodiment of the pilot balloon assembly 40 having a suitable pilot balloon guard 43. In this embodiment, a rounded cylindrical enclosure 84 couples to the pilot line 38 and to a luer guard protector 86 provided for the pilot valve 62. In the illustrated embodiment, the enclosure 84 is made of two substantially similar pieces. One piece 88 is shown in more detail in FIG. 6. As shown, the piece 88 includes features that enable the piece 88 to mate with a corresponding piece via apertures 90 and teeth 92. More specifically, the teeth 92 of the piece 88 are configured to be received by apertures of another piece, and the apertures 90 of the piece 88 are configured to receive teeth of another piece. In this way, the two halves of the enclosure 84 may fit together to enclose the pilot valve balloon 41.

It should be noted that in designs that include two halves, the two halves may fit together in a variety of ways. For example, piece 88 may fit with a corresponding piece via a snap fit, a press fit, a bonded fit, or via any other securement mechanism. Additionally, the guard 43 may be coupled to the tracheal tube assembly via a press fit, a snap fit, a bonded fit, molding with the luer guard, or any other desired mechanism. Still further, it should be noted that the walls of the pilot balloon guard 43, such as walls 84, may be semi-transparent, transparent, or translucent to enable the clinician or user to view the pilot balloon 41 when the guard 43 is in use.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A tracheostomy tube assembly, comprising:
an outer cannula comprising an open distal end and a proximal end, the open distal end being configured to be inserted into an airway of a patient;
an inner cannula configured to be inserted into the outer cannula;
a flange member disposed about the proximal end of the outer cannula;
a connector coupled to the proximal end of the outer cannula, wherein the inner cannula and the connector form a contiguous passageway for exchanging fluid with the airway of the patient in operation;
a cuff disposed around the outer cannula above the open distal end and configured to be inflated to seal the cuff against the walls of the patient's airway; and
a pilot balloon assembly comprising a pilot balloon configured to inflate when air is supplied to the cuff to indicate the state of inflation of the cuff, and further comprising a pilot balloon guard disposed about the pilot balloon during operation to prevent transfer of compressive forces to the pilot balloon.

2. The assembly of claim 1, wherein the pilot balloon assembly further comprises an inflation lumen coupled to the pilot balloon and configured to transfer air to the cuff to inflate the cuff.

3. The assembly of claim 1, wherein the pilot balloon guard comprises a first piece having at least one extension and a second piece having at least one aperture for receiving the at least one extension to couple the first piece and the second piece.

4. The assembly of claim 3, wherein the first piece and the second piece are formed via injection molding.

5. The assembly of claim 1, wherein the pilot balloon guard comprises a single injection molded piece.

6. The assembly of claim 1, wherein the pilot balloon guard is at least partially formed from polycarbonate.

7. The assembly of claim 1, wherein the pilot balloon guard comprises at least one venting aperture to enable equalization between the pressure in the volume between the pilot balloon guard and the external environment.

8. The assembly of claim 1, wherein the pilot balloon guard comprises a clam shell comprising a first half and a second half substantially similar to the first half and coupled to the first half with a hinge.

9. The assembly of claim 1, comprising a conduit coupled to the connector for connecting to a at least one of an airway accessory, a ventilator, a humidifier, or a combination thereof.

10. A pilot balloon assembly for a tracheal tube, comprising:
a pilot balloon guard comprising an enclosure having walls that define an interior volume that is greater than an interior volume of a pilot balloon configured to be received by the enclosure and to inflate within the enclosure to indicate inflation of a cuff of the tracheal tube, wherein the enclosure absorbs externally applied forces to prevent transfer of the externally applied forces to the pilot balloon.

11. The assembly of claim 10, comprising an inflation line coupled to the pilot balloon and to the cuff and configured to enable transfer of air to the cuff for inflation and to the pilot balloon for indication of cuff inflation.

12. The assembly of claim 11, wherein the enclosure is configured to attach to the inflation line and to remain attached to the inflation line when placed in an open position.

13. The assembly of claim 10, wherein the enclosure comprises one or more apertures configured to enable transfer of air between the external environment and the interior volume.

14. The assembly of claim 10, wherein the enclosure comprises a transparent or translucent polymer.

15. The assembly of claim 10, wherein the pilot balloon comprises a neonatal or pediatric sized pilot balloon configured for use with a neonatal or pediatric tracheal tube.

16. The assembly of claim 10, wherein the walls of the enclosure comprise reinforcing ribs.

17. The assembly of claim 10, wherein the walls of the enclosure are configured to withstand forces up to approximately 30 kilograms.

18. A tracheal tube assembly, comprising:
a cannula comprising a distal end and a proximal end, the distal end being configured to be inserted into a patient's trachea;
an inflatable cuff disposed about the proximal end of the cannula and configured to be inflated and deflated to seal against a wall of the patient's airway;
an inflation lumen disposed in a wall of the cannula and configured to facilitate the transfer of air to the inflatable cuff;
a pilot balloon coupled to the inflation lumen and configured to inflate when air is supplied to the cuff to indicate the state of inflation of the cuff; and
a pilot balloon guard comprising a casing configured to enclose the pilot balloon, wherein the pilot balloon guard absorbs externally applied forces during operation to prevent transfer of the externally applied forces to the pilot balloon.

19. The assembly of claim 18, wherein the casing of the pilot balloon guard comprises a first piece having at least one extension and a second piece having at least one aperture for receiving the at least one extension to couple the first piece to the second piece.

20. The assembly of claim 18, wherein the pilot balloon comprises a neonatal or pediatric sized pilot balloon configured for use with a neonatal or pediatric tracheal tube.
